(19) United States
(12) Patent Application Publication
(10) Pub. No.: US 2022/0268775 A1
(43) Pub. Date: Aug. 25, 2022

(54) SOLUBLE PROGRAMMED CELL DEATH PROTEIN-1 AS A BIOMARKER IN CANCER PATIENTS

(71) Applicant: AiVita Biomedical, Inc., Irvine, CA (US)

(72) Inventors: Robert O. Dillman, Irvine, CA (US); Gabriel Nistor, Irvine, CA (US)

(21) Appl. No.: 17/631,200

(22) PCT Filed: Jul. 31, 2020
(86) PCT No.: PCT/US2020/044492

§ 371 Date: Jan. 28, 2022

Related U.S. Application Data
(60) Provisional application No. 62/881,169, filed on Jul. 31, 2019.

(51) Int. Cl. G01N 33/574; A61K 39/00

(52) U.S. Cl. CPC G01N 33/5743; A61K 39/0011; G01N 2333/70596; A61K 2039/5152; A61K 2039/5154; A61K 2039/876

(57) ABSTRACT

Disclosed herein are methods for selecting cancer patients for autologous cancer vaccine therapy and methods for predicting survival of cancer patients after autologous cancer vaccine therapy comprising, measuring the level of soluble programmed cell death protein-1 (sPD-1) in the blood of the patient.

### RELATED APPLICATIONS

This application claims the benefit of U.S. Provisional Application No. 62/881,169, filed Jul. 31, 2019, the entire contents of which are incorporated by reference herein.

### BACKGROUND

Programmed cell death protein-1 (PD-1; CD279) is expressed on the surface membrane of immune cells, and has a role in regulating immune responses via interaction with specific ligands (PD-L1 and PD-L2). More recently it has been discovered that there is also a soluble form of PD-1 and, because of the role of membrane-bound form of PD-1 as an immune checkpoint, there has been speculation that levels of soluble PD-1 (sPD-1) could be useful as a prognostic, diagnostic, or therapeutic biomarker or predictive biomarker in cancer patients.

### SUMMARY

Soluble PD-1 is upregulated on activated lymphocytes by interferon gamma, during a Th1 immune response, and can therefore be used as a prognostic biomarker or predictive biomarker in cancer patients treated with vaccines. In cancer patients, very low levels of sPD-1 may indicate lack of an existing anti-cancer immune response while very high levels may indicate an active immune response that has been suppressed. In between these extremes, a decrease in PD-1 following injections of an anti-cancer vaccine may indicate an enhanced immune response that has not been suppressed.

Disclosed herein are methods for selecting cancer patients for cancer vaccine therapy comprising measuring the level of soluble programmed cell death protein-1 (sPD-1) in the blood of the patient, wherein levels of sPD-1 less than about 500 pg/ml are predictive of efficacy of the cancer vaccine therapy. In some embodiments, the therapeutic cancer vaccine is an autologous vaccine. In some embodiments, the therapeutic cancer vaccine is a dendritic cell vaccine.

Also disclosed herein are methods for predicting survival of cancer patients after cancer vaccine therapy comprising, measuring the level of soluble programmed cell death protein-1 (sPD-1) in the blood of the patient, wherein levels of sPD-1 are:

(i) less than about 100 pg/ml before onset of therapy; or

(ii) less than about 200,000 pg/ml before onset of therapy and less after three administrations or doses of cancer vaccine therapy than before onset of therapy.

Also disclosed are methods for treatment of cancer in a subject in need thereof, comprising measuring the level of soluble programmed cell death protein-1 (sPD-1) in the blood of the patient; and administering cancer vaccine if the levels of sPD-1 are less than about 500 pg/ml.

Also disclosed are methods for treatment of cancer in a subject in need thereof, comprising measuring the level of sPD-1 in the blood of the patient before onset of cancer vaccine therapy; administering one or more rounds of cancer vaccine therapy to the patient; measuring the level of sPD-1 in the blood of the patient after the one or more doses of cancer vaccine therapy; and administering one or more additional rounds of cancer vaccine if the levels of sPD-1 have decreased after one or more rounds of cancer vaccine therapy. In various embodiments, a round may comprise one, two, or three administrations of the vaccine. In aspects of these embodiments, the levels of sPD-1 are less than about 200,000 pg/ml before onset of therapy.

In some embodiments, the cancer is melanoma.

For all references to a cancer vaccine or cancer vaccine therapy, in some embodiments, the cancer vaccine is a dendritic cell vaccine; in some embodiments the cancer vaccine is a tumor cell vaccine. In a further aspect of any of these embodiments, the cancer vaccine is an autologous vaccine. Some embodiments specifically require one or more of these features. Other embodiments specifically exclude one or more of these features.

With respect to the various methods of treatment, if a subject does not meet the criteria for initiating or continuing cancer vaccine therapy according to any of the herein disclosed embodiments, then in a further aspect of the embodiment, the subject is treated with an immune checkpoint inhibition therapy instead of the vaccine.

### BRIEF DESCRIPTION OF THE DRAWINGS

FIG. 1: Changes in sPD-1 between week-0 and week-4 for individual patients receiving cancer vaccines. (FIG. 1A) shows all 39 patients by treatment arm—dendritic cell vaccine (DCV) or tumor cell vaccine (TCV); (FIG. 1B) shows the 23 patients who survived less than three years by treatment arm (DCV or TCV); and (FIG. 1C) shows the 16 patients who survived more than three years by treatment arm (DCV or TCV).

FIG. 2: Mean levels of baseline sPD-1 in various cohorts of patients receiving cancer vaccines. (FIG. 2A) shows there was no difference in sPD-1 levels (Mann-Whitney U Test) by survival less than three years versus greater than three years, or DCV versus TCV treatment arm. (FIG. 2B) shows a trend toward higher sPD-1 levels in DCV-treated patients who survived less than three years compared to those who survived more than three years (p=0.131 Mann-Whitney U-Test) This was not observed in the TCV-treated patients (p=0.857 Mann-Whitney U-Test). DCV=dendritic cell vaccine, TCV=tumor cell vaccine. Data are presented as mean±SD.

FIG. 3: Survival of patients having received cancer vaccines based on whether baseline sPD-1 levels were above or below 1,200 pg/mL. (FIG. 3A) shows the distribution of survival for the 19 patients who had a baseline sPD-1 level less than 1,200 pg/mL, and for the 20 patients who had a baseline sPD-1 level greater than 1,200 pg/mL. The proportion of patients who survived more than three years, 9/19 vs 7/20, did not differ (p=0.523, Fisher Exact Test). (FIG. 3B) shows actual survival curves (all patients followed to death or five years with none lost to follow-up). There was no difference between the curves (p=0.453, Mantel-Haenszel log rank-test).

FIG. 4: Survival of patients having received cancer vaccines by whether sPD-1 increased or decreased between week-0 and week-4. (FIG. 4A) shows distribution of survival with mean and standard deviation bars for the 14 patients whose sPD-1 decreased and for the 25 patients whose sPD-1 increased between week-0 and week-4. The proportion of patients who survived more than three years, 5/14 vs 11/25, did not differ (p=0.740, Fisher Exact Test). (FIG. 4B) shows the actual survival curves (all patients followed to death or five years with none lost to follow-up). There was no difference between the curves (p=0.624, Mantel-Haenszel log rank-test).

FIG. 5: Survival distribution for all patients having received cancer vaccines by treatment. The 39 patients were grouped by four different ranges of baseline sPD-1 levels. There was no difference in survival based on week-0 baseline sPD-1 ranges. Medians for all patients in each sPD-1 range are shown by black bar. Survivals for individual patients are shown for each of the 17 DCV-treated (circles) and each of the 22 TCV-treated patients (squares). For DCV-treated patients who had baseline sPD-1 levels less than 200,000 pg/mL, 15/15 survived more than one year compared to 0/2 who had baseline sPD-1 levels greater than 200,000 pg/mL (p=0.0074, Fisher Exact Test). For TCV-treated patients who had baseline sPD-1 levels less than 200,000 pg/mL, 14/20 survived more than one year compared to 2/2 who had sPD-1 levels greater than 200,000 at baseline (p=1.00 Fisher Exact Test). For DCV-treated patients who had baseline sPD-1 levels less than 500 pg/mL, 6/6 survived more than one year compared to 9/11 DCV-treated patients who had baseline sPD-1 levels greater than 500 pg/mL (p=0.515, Fisher Exact Test). For TCV-treated patients who had baseline sPD-1 levels less than 500 pg/mL, 6/8 survived more than one year compared to 10/14 who had sPD-1 levels greater than 200,000 pg/mL at baseline (p=1.00 Fisher Exact Test).

FIG. 6: sPD-1 levels and survival of metastatic melanoma patients treated with autologous dendritic cell vaccine. Survival is shown by individual patients displaying mean with standard deviation (FIG. 6A) and actual survival curves (FIG. 6B) for two cohorts of DCV-treated patients. All patients were followed to death or for five years with none lost to follow-up. The two cohorts were defined by baseline levels of sPD-1 and changes in sPD-1 levels between week-0 and week-4. In DCV-treated patients, having 1) sPD-1 level less than 100 pg/mL at baseline, or 2) sPD-1 less than 200,000 pg/mL and a decrease in sPD-1 from week-0 to week-4 (sPD-1 low/decreased), was predictive of longer survival compared to sPD-1 level of greater than 200,000 pg/mL or greater than 100 pg/mL and an increase in sPD-1 between week-0 and week-4 (sPD-1 high/increased). Survival of autologous dendritic cell vaccine-treated metastaic melanoma patients was greater than 3 years in 8/9 vs 2/8 of patients (dot plot—p=0.0152, Fisher Exact Test) and the median overall survival (OS) was 48.8 vs 17.6 months (survival curve—p=0.182, Mantel-Haenszel log rank test).

FIG. 7: sPD-1 and survival of metastatic melanoma patients treated with autologous tumor cell vaccine. Survival is shown by individual patients displaying mean with standard deviation (FIG. 7A), and actual survival curves (FIG. 7B) for two cohorts of TCV-treated patients. All patients were followed to death or for five years with none lost to follow up. The two cohorts were defined by baseline levels of sPD-1 and changes in sPD-1 levels between week-0 and week-4. In TCV-treated patients, having 1) sPD-1 level less than 100 pg/mL at baseline, or 2) sPD-1 less than 200,000 pg/mL and a decrease in sPD-1 from week 0 to week 4 (sPD-1 low/decreased), was not predictive of longer survival compared to sPD-1 level of greater than 200,00 pg/mL or greater than 100 pg/mL and an increase in sPD-1 between week-0 and week-4 (sPD-1 high/increased). Survival of autologous tumor cell vaccine-treated metastaic melanoma patients was greater than 3 years in 5/14 vs 1/8 of patients (dot plot—p=0.351 Fisher Exact Test) and median OS was 30.3 vs 16.9 mos (survival curve—p=0.791, Mantel-Haenszel log rank test).

### DETAILED DESCRIPTION

Because of its role as an immune checkpoint, levels of soluble programmed cell death protein-1 (sPD-1) could be useful as a prognostic biomarker or predictive biomarker in cancer patients treated with vaccines. Very low levels of sPD-1 may indicate lack of an existing anti-cancer immune response. Conversely, very high levels may indicate an active immune response that is suppressed. In between these extremes, a decrease in PD-1 following injections of an anti-cancer vaccine may indicate an enhanced immune response that has not been suppressed.

There have been a few reports of sPD-1 levels in various cancers with no consistent prognostic or predictive patterns. In one study, higher levels of sPD-1 were associated with longer progression-free survival and overall survival (OS) in 38 patients with non-small cell lung cancer who were being treated with erlotinib, an inhibitor of mutated epidermal growth factor receptor (Sorensen et al. Lung Cancer 100:77-84, 2016). Among patients with hepatitis B virus, those who had an elevated sPD-1 had a higher risk of developing hepatocellular cancer (Li et al. Oncotarget 18:46020-33, 2017). Among 120 patients with hepatocellular cancer, those who had higher sPD-1 levels had a worse prognosis (Chang et al. Cancer Immunol Immunother 68:353-63, 2019). In 41 patients with advanced pancreatic cancer, sPD-1 levels were not correlated with outcome (Kruger et al. Oncoimmunol 6:e1310358, 2017). In that study, levels of sPD-1 and sPD-L1 were closely correlated and elevated in association with an elevated C-reactive protein, suggestive of systemic inflammation, and tumor infiltration with T cells, which was consistent with an active anti-tumor immune response that was being blunted. In that trial, the median sPD-1 level was only 117 pg/mL with a range from 40 to 26,000 pg/mL using an ELISA with chemiluminescent detection. While sPD-1 was detectable in the serum of all patients analyzed, sPD-L1 was below the lower limit of detection in 15/41 patients.

The present disclosure is directed to the use of sPD-1 as an immune marker in patients with metastatic melanoma who were enrolled in a randomized phase II trial testing autologous dendritic cell vaccines (DCV) and autologous tumor cell vaccines (TCV). In particular, sPD-1 may provide a surrogate marker that might reflect immune response and association with survival in patients treated with these vaccines. For both tumor cell vaccine (TCV) and dendritic cell vaccine (DCV) compositions, the antigen source was irradiated autologous cancer cells from short-term cell cultures derived from surgically excised autologous tumor. The following points were evaluated experimentally. (1) was baseline sPD-1 prognostic for survival in these patients with metastatic melanoma, or for either of the two treatment-defined cohorts; (2) within each treatment-defined cohort, was either vaccine efficacious in patients with very low sPD-1 levels and/or very high sPD-1 levels; (3) if there was a change in sPD-1 from week-0, one week before the first of three weekly vaccine injections, to week-4, one week after the third injection, was this predictive of survival for all patients or in either treatment-defined cohort; and (4) could sPD-1 be used to define cohorts that were prognostic or predictive of survival.

Blood samples obtained during a randomized trial in patients with metastatic melanoma were tested from 22 patients treated with a TCV and 17 treated with a DCV. Survival was improved in DCV-treated patients. sPD-1 was measured at week-0, one week before the first of three weekly subcutaneous injections, and at week-4, one week after the third injection. The combination of a very low baseline sPD-1, or absence of a very high PD-1 at baseline followed by a decline in sPD-1 at week-4, was predictive of surviving three or more years in DCV-treated patients, but not TCV-treated patients. Among DCV-treated patients, these sPD-1 criteria appropriately classified 8/10 (80%) of 3-year survivors, and 6/7 (86%) of patients who did not survive three years. These observations suggest that sPD-1 may be a useful biomarker for melanoma patients being considered for treatment with DCV vaccines, and/or to predict efficacy.

Disclosed herein are methods for selecting cancer patients for cancer vaccine therapy comprising, measuring the level of sPD-1 in the blood of the patient, wherein levels of sPD-1 less than about 500 pg/ml are predictive of efficacy of the cancer vaccine therapy. In some embodiments, the level of sPD-1 is less than 250 pg/ml. In some embodiments, the level of sPD-1 is less than 100 pg/ml. In some of these embodiments, the cancer vaccine is an autologous cancer vaccine. Some embodiments further comprise administering a cancer vaccine if the pre-treatment level of sPD-1 is less than a threshold level of sPD-1. In some aspects of these embodiments the threshold level is 500, 250, or 100 pg/ml. Some embodiments further comprise administering an immune checkpoint inhibition therapy if the level of sPD-1 is greater than threshold level of sPD-1. In some aspects of these embodiments the threshold level is 500, 550, or 600 pg/ml.

Further disclosed are improved methods of treating cancer patients with therapeutic cancer vaccines. In some embodiments, the cancer patient is administered the vaccine if the level of sPD-1 in their blood is less than a threshold level. Some embodiments further comprise administering an immune checkpoint inhibition therapy if the level of sPD-1 in their blood is not less than the threshold level. In various aspects of these embodiments the threshold level of sPD-1 is 500 pg/ml, 250 pg/ml, or 100 pg/ml. In some of these embodiments the vaccine is a tumor cell vaccine. In some of these embodiments, the vaccine is a dendritic cell vaccine. In some of these embodiments, the cancer vaccine is autologous. Some embodiments specifically include a particular threshold level or type of vaccine. Some embodiments specifically exclude a particular threshold level or type of vaccine.

Further disclosed are improved methods of cancer vaccine therapy. In some embodiments, a patient with a level of sPD-1 less than 200,000 pg/ml prior to initiation of vaccine therapy (or a round of vaccine therapy) is only administered a further dose of the vaccine if the level of sPD-1 has decreased from its level before the onset of therapy (or onset of a round of therapy) after a certain number of administrations of the vaccine. In various aspects of these embodiments, the number of administrations is one, two, or three. In further embodiments, such evaluation may be conducted iteratively after each round of one, two, or three administrations. In some of these embodiments the vaccine is a tumor cell vaccine. In some of these embodiments, the vaccine is a dendritic cell vaccine. In some of these embodiments, the cancer vaccine is autologous. Some embodiments specifically include a particular number of administrations per round of therapy or type of vaccine. Some embodiments specifically exclude a particular number of administrations per round of therapy or type of vaccine. In some embodiments, if the level of sPD-1 has not decreased from its level before the onset of therapy (or round of therapy) after a certain number of administrations of the vaccine, the patient is switched to immune checkpoint inhibition therapy.

Also disclosed herein are methods for predicting survival of cancer patients after autologous cancer vaccine therapy comprising, measuring the level of sPD-1 in the blood of the patient, wherein levels of sPD-1 are:

(i) less than about 100 pg/ml before onset of therapy; or

(ii) less than about 200,000 pg/ml before onset of therapy and decreased after three administrations or doses of autologous cancer vaccine therapy from the level before onset of therapy.

In some embodiments, the cancer is melanoma, lung cancer, breast cancer, colorectal cancer, pancreatic cancer, liver cancer, skin cancer, head and neck cancer, lymphoma, kidney cancer, or prostate cancer. In some embodiments, the cancer is melanoma.

In some embodiments, the cancer vaccine is a dendritic cell vaccine. In other embodiments, the cancer vaccine is a tumor cell vaccine. In any of these embodiments, the vaccine may be autologous.

Levels of sPD-1 can be measured by any assay known to persons of ordinary skill in the art.

Immune checkpoint inhibition therapy refers to the use of pharmaceuticals, typically biologics, that act on regulatory pathways in the differentiation and activation of T cells to promote the passage of a T cell developmental program through these checkpoints so that anti-tumor (or other therapeutic) activity can be realized. The agents bringing about immune checkpoint therapy are commonly called immune checkpoint inhibitors and it should be understood that it is the check on T cell development that is being inhibited. Thus, while many immune checkpoint inhibitors also inhibit the interaction of receptor-ligand pairs (e.g., programmed cell death 1 (PD-1) interaction with programmed death-ligand 1 (PD-L1)), other checkpoint inhibitors (such as anti-OX40 and anti-ICOS) act as agonists of targets that release or otherwise inhibit the check on T cell development, ultimately promoting effector function and/or inhibiting regulatory function. Most commonly, antibodies against one member of the receptor-ligand pair are used. In alternative embodiments, the antibody is replaced with another protein that similarly binds to the immune checkpoint target molecule. In some instances, these non-antibody molecules comprise an extracellular portion of the immune checkpoint target molecule's ligand or binding partner, that is, at least the extracellular portion needed to mediate binding to the immune checkpoint target molecule. In some embodiments, this extracellular binding portion of the ligand is joined to additional polypeptide in a fusion protein. In some embodiments, the additional polypeptide comprises an Fc or constant region of an antibody.

In some embodiments, immune checkpoint inhibition therapy specifically refers to inhibition of the PD-1 checkpoint. In some aspects of these embodiments, the inhibitor comprises a PD-1 antagonist, such as an anti-PD-1 antagonistic antibody, of binding fragment thereof. In some aspects of these embodiments, the inhibitor comprises a PD-L1 antagonist, such as an anti-PD-L1 antagonistic antibody, of binding fragment thereof. Some embodiments specifically exclude one or another of these reagents or any other immune checkpoint inhibitor.

Programed death-1 (PD-1) is a checkpoint protein on T cells. Antibodies against both PD-1 and its binding partner programmed death-ligand 1 (PD-L1) have been used clinically as immune checkpoint inhibitors (PD-1 blockade). Examples of monoclonal antibodies that target PD-1/PL-L1 include the anti-PD-1 mAbs nivolumab (OPDIVO®, Bristol-Myers Squibb), pembrolizumab (KEYTRUDA®, Merck & Co.), cemiplimab-rwlc (LIBTAYO®, Regeneron Pharmaceuticals) and the anti-PD-L1 mAbs durvalumab (MED14736, IMFINZI™, Medimmune), atezolizumab (MPDL3280A; TECENTRIQ®, Hoffman-La Roche), avelumab (BAVENCIO®, EMD Serono), and BMS-936559 (Bristol-Myers Squibb). These may be referred to as means for PD-1 blockade, means for inhibiting PD-1/PD-L1 binding, or means for immune checkpoint inhibition. Some embodiments specifically include one or more of these reagents. Other embodiments specifically exclude one or more of these reagents.

The term “treating” or “treatment” broadly includes any kind of treatment activity, including the diagnosis, assessment of risk or prognosis, mitigation, or prevention of disease, or aspect thereof, in man or other animals, or any activity that otherwise affects the structure or any function of the body of man or other animals. Treatment activity includes the administration of the medicaments, dosage forms, and pharmaceutical compositions described herein to a patient, especially according to the various methods of treatment disclosed herein, whether by a healthcare professional, the patient his/herself, or any other person. Treatment activities include the orders, instructions, and advice of healthcare professionals such as physicians, physician's assistants, nurse practitioners, and the like, that are then acted upon by any other person including other healthcare professionals or the patient him/herself. This includes, for example, direction to the patient to undergo, or to a clinical laboratory to perform, a diagnostic procedure, such as an assessment of sPD-1 level as disclosed herein so that ultimately the patient may receive the benefit thereof. In some embodiments, the orders, instructions, and advice aspect of treatment activity can also include encouraging, inducing, or mandating that a particular medicament or test, or combination thereof, be chosen for treatment of a condition—and the medicament is actually used—by approving insurance coverage for the medicament or test, denying coverage for an alternative medicament or test, including the medicament or test on, or excluding an alternative medicament or test, from a drug formulary, or offering a financial incentive to use the medicament or test, as might be done by an insurance company or a pharmacy benefits management company, and the like. In some embodiments, treatment activity can also include encouraging, inducing, or mandating that a particular medicament or test be chosen for treatment of a condition—and the medicament is actually used—by a policy or practice standard as might be established by a hospital, clinic, health maintenance organization, medical practice or physicians group, and the like. All such orders, instructions, and advice are to be seen as conditioning receipt of the benefit of the treatment on compliance with the instruction. In some instances, a financial benefit is also received by the patient for compliance with such orders, instructions, and advice. In some instances, a financial benefit is also received by the healthcare professional for compliance with such orders, instructions, and advice.

### EXAMPLES

### Example 1

Patients and Blood Samples

Blood samples were obtained from metastatic melanoma patients who were enrolled in a randomized phase II trial of an autologous tumor cell vaccine (TCV) and an autologous dendritic cell vaccine (DCV) (NCT00948480). The trial was conducted per the doctrine of Helsinki and all subjects gave written informed consent to participate. The details regarding the vaccine products and patient outcomes were previously published (Dillman et al. J. Immunother. 35:641-649, 2012; Dillman et al. J. Immunother Cancer 6:19, 2018 https:doi.org/10.1186/s40425-018-03330-1, both of which are incorporated by reference herein in their entirety). Briefly, the TCV consisted of irradiated self-renewing cancer cells, and the DCV consisted of autologous dendritic cells loaded with antigens from self-renewing autologous cancer cells. Such cancer cells have characteristics of tumor-initiating cells including cancer stem cells and progenitor cells. Dendritic cells (DC) were derived from peripheral blood mononuclear cells obtained during a leukapheresis procedure. Both cellular vaccines were suspended in a granulocyte-macrophage colony stimulating factor (GM-CSF)-containing vehicle for subcutaneous injections that were planned for weeks 1, 2, 3, 8, 12, 16, 20, and 24.

The eligibility criteria, patient characteristics, early survival, and long-term survival outcomes for the patients enrolled in the randomized trial have been published [Dillman et al., 2012; Dillman et al., 2018]. Briefly, patients had to have stage 4 or recurrent stage 3 melanoma, had undergone resection of one or more lesions from which a short-term cell line had been established, had a Karnofsky performance status of 70 or greater at the time of randomization, and were referred by their managing physician for randomization to TCV or DCV. Forty-two patients were randomized into the study. Long-term follow up of the randomized trial confirmed that DCV was associated with longer median survival (43.4 vs 20.5 months), better actual survival at three years (61% vs 25%, p=0.018), and a 70% reduction in the risk of death (p=0.0053).

As a component of the trial, patients had blood samples collected one week before starting the vaccine injections and one week after the third weekly injection. Data from patients for whom blood samples were available from both week-0, one week prior to the first injection, and week-4, one week following the third injection are discussed herein. Paired samples were available for 39 of the 42 patients. The three patients for whom blood samples were missing were two TCV-treated patients who did not have week-4 blood samples collected because of rapidly progressive disease, and one DCV-treated patient who survived more than five years, but rescinded permission to test his blood. Even without the survival data from those three patients, the proportion surviving three years was still greater among DCV-treated patients than TCV-treated patients (10/17 versus 6/22, p=0.047, Fisher Exact Test).

Soluble PD-1 Levels

Paired cryopreserved serum samples from week-0 and week-4 from each patient were analyzed using a quantitative, multiplex, enzyme-linked immunosorbent assay per good laboratory practice (GLP) standards (Raybiotech, Inc., Norcross, Ga.). Results for sPD-1 were reported in picograms/milliliter (pg/mL).

Statistics

Proportions were compared using the Fisher exact test. Because of the broad data distribution, grouped sPD-1 levels were compared by the non-parametric Mann-Whitney U test. Paired comparisons of week-0 versus week-4 sPD-1 levels were made by the Wilcoxon signed-rank test. Differences in survival curves were compared using the Mantel-Haenszel log-rank test. For all tests, the significance level was 0.05 and hypothesis tests were two-tailed.

Results

Summary Data

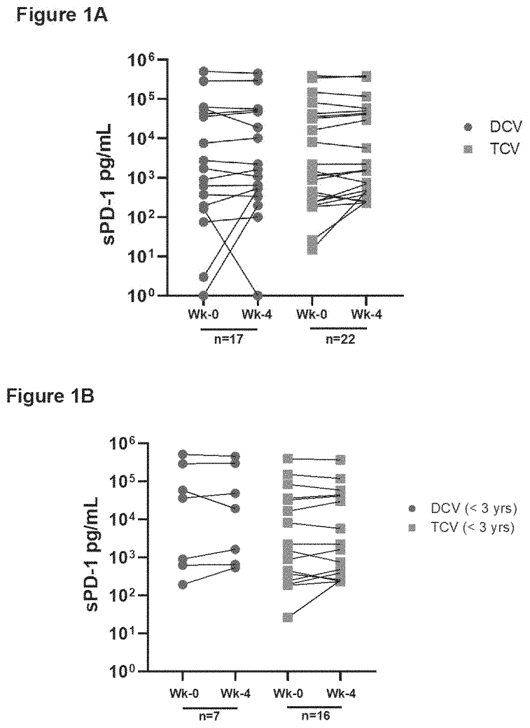

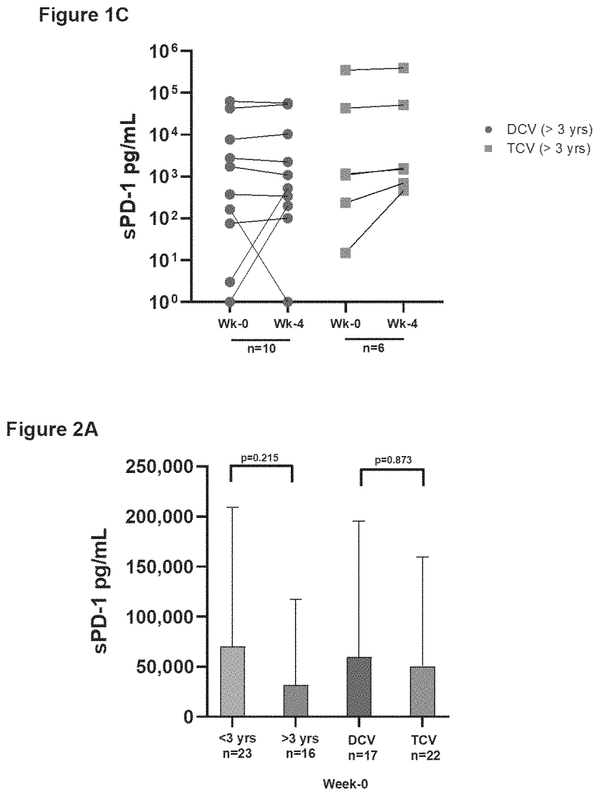

Tables 1 and 2 depict the sPD-1 data for all 39 patients. Table 1 displays the week-0 baseline sPD-1, week-4 sPD-1, absolute, and percentage changes in sPD-1 from week-0 baseline to week-4, and actual survival for the DCV-treated patients. Table 2 displays similar data for TCV-treated patients. In addition, FIG. 1 shows the changes in sPD-1 levels graphically for each individual patient by treatment arm (FIG. 1A), and by subcohorts based on 3-year survival within each group (FIGS. 1B and 1C). Table 3 shows the median and mean sPD-1 levels for week-0 and week-4 and changes in the mean and median sPD-1 levels for all 39 patients as a group, as well as cohorts defined by treatment (DCV or TCV) and/or survival (greater or less than three years).

TABLE 1
Autologous dendritic cell vaccine (DCV): sPD-1 levels
before and after three weekly injections of DCV and associated
survival in patients with metastatic melanoma.

| | Week-0 | Week-4 | Change in | | |
|---|---|---|---|---|---|
| Patient | sPD-1 | sPD-1 | sPD-1 | | Survival |
| Number | (pg/mL) | (pg/mL) | (pg/mL) | % Change | (months) |
| 1 | 1 | 198 | 198 | 19,700.0% | 42.2 |
| 2 | 3 | 520 | 517 | 17,233.3% | 44.6 |
| 3 | 75 | 100 | 25 | 34.1% | 60+ |
| 4 | 164 | 0 | −164 | −100.0% | 60+ |
| 5 | 191 | 532 | 341 | 178.5% | 19.1 |
| 6 | 372 | 335 | −37 | −9.9% | 52.9 |
| 7 | 618 | 650 | 32 | 5.2% | 25.2 |
| 8 | 897 | 1,613 | 716 | 79.8% | 13.0 |
| 9 | 1,733 | 1,078 | −655 | −37.8% | 53.0 |
| 10 | 2,723 | 2,226 | −497 | −18.2% | 60+ |
| 11 | 7,645 | 10,340 | 2,695 | 35.3% | 60+ |
| 12 | 36,277 | 48,001 | 11,724 | 32.3% | 17.6 |
| 13 | 42,393 | 52,570 | 10,177 | 24.0% | 60+ |
| 14 | 57,687 | 19,160 | −38,527 | −66.8% | 18.6 |
| 15 | 62,777 | 56,841 | −5,936 | −9.5% | 38.6 |
| 16 | 290,060 | 298,072 | 8,012 | 2.8% | 7.9 |
| 17 | 511,063 | 455,212 | −55,851 | −10.9% | 9.6 |
| 18 | — | — | — | — | 60+ |

sPD-1 = soluble PD-1;
pg/mL = picogram/milliliter

TABLE 2
Autologous tumor cell vaccine (TCV): sPD-1 levels before
and after three weekly injections of TCV and associated
survival in patients with metastatic melanoma.

| | Week-0 | Week-4 | Change in | | |
|---|---|---|---|---|---|
| Patient | sPD-1 | sPD-1 | sPD-1 | | Survival |
| Number | (pg/mL) | (pg/mL) | (pg/mL) | % change | (months) |
| 1 | 15 | 462 | 447 | 96.8% | 60+ |
| 2 | 26 | 247 | 221 | 89.5% | 30.3 |
| 3 | 183 | 230 | 47 | 20.4% | 4.0 |
| 4 | 193 | 388 | 195 | 50.3% | 9.3 |
| 5 | 233 | 694 | 461 | 66.4% | 60+ |
| 6 | 238 | 481 | 243 | 50.5% | 13.2 |
| 7 | 364 | 259 | −105 | −40.5% | 33.7 |
| 8 | 446 | 234 | −212 | −90.6% | 32.2 |
| 9 | 877 | 1,591 | 714 | 44.9% | 3.9 |
| 10 | 1,075 | 1,584 | 509 | 32.1% | 60+ |
| 11 | 1,152 | 1,488 | 336 | 22.6% | 60+ |
| 12 | 1,509 | 745 | −764 | −102.6% | 9.0 |
| 13 | 2,201 | 2,238 | 37 | 1.7% | 16.9 |
| 14 | 8,121 | 5,710 | −2,411 | 95.3% | 9.9 |
| 15 | 16,277 | 29,276 | 12,999 | −42.2% | 14.6 |
| 16 | 32,287 | 41,123 | 8,836 | 44.4% | 21.7 |
| 17 | 35,637 | 43,826 | 8,189 | 21.5% | 2.5 |
| 18 | 43,095 | 50,939 | 7,844 | 18.7% | 60+ |
| 19 | 82,755 | 58,223 | −24,532 | 15.4% | 19.9 |
| 20 | 150,588 | 118,159 | −32,429 | −27.4% | 32.3 |
| 21 | 343,919 | 389,327 | 45,408 | 11.7% | 60+ |
| 22 | 392,201 | 364,014 | −28,187 | −7.7% | 21.1 |
| 23 | — | — | — | — | 0.7 |
| 24 | — | — | — | — | 1.1 |

TABLE 3
sPD-1 levels in metastatic melanoma patients enrolled in a randomized trial.

| | | | | | Change | Change |
|---|---|---|---|---|---|---|
| | | Median | Mean | St Dev | Mean Wk-0 | Median Wk-0 |
| | Number of | sPD-1 | sPD-1 | sPD-1 | to Wk-4 | to wk-4 |
| Population | Patients | (pg/mL) | (pg/mL) | (pg/mL) | (pg/mL) | (pg/mL) |
| All Wk-0 | N = 39 | 1,509 | 54,566 | 119,845 | −1779 | +82 |
| All Wk-4 | N = 39 | 1,591 | 52,787 | 115,284 | (−3.3%) | (+5.4%) |

>3 yr survival

| | | | | | | |
|---|---|---|---|---|---|---|
| Wk-0 | n = 16 | 1,114 | 31,711 | 85,580 | +3,833 | +170 |
| Wk-4 | n = 16 | 1,283 | 35,544 | 96,663 | (+12.1%) | (+15.2%) |

<3 yr survival

| | | | | | | |
|---|---|---|---|---|---|---|
| Wk-0 | n = 23 | 2,201 | 70,465 | 138,459 | −5,683 | +37 |
| Wk-4 | n = 23 | 2,238 | 64,782 | 127,312 | (−8.1%) | (+1.7%) |

Treatment arm

| | | | | | | |
|---|---|---|---|---|---|---|
| TCV Wk-0 | n = 22 | 1,331 | 50,609 | 109,063 | −98 | +257 |
| TCV Wk-4 | n = 22 | 1,588 | 50,511 | 109,634 | (−0.2%) | (+19.3%) |
| DCV Wk-0 | n = 17 | 1,733 | 59,687 | 135,833 | −3,955 | −120 |
| DCV Wk-4 | n = 17 | 1,613 | 55,732 | 125,510 | (−6.6%) | (−6.9%) |

Treatment arm and survival

| | | | | | | |
|---|---|---|---|---|---|---|
| TCV OS | <3 yrs | | | | | |
| Wk-0 | n = 16 | 1,855 | 45,244 | 101,076 | −3,572 | +60 |
| Wk-4 | n = 16 | 1,915 | 41,672 | 91,864 | (−7.9%) | (+3.2%) |
| TCV OS | >3 yrs | | | | | |
| Wk-0 | n = 6 | 1,114 | 64,915 | 137,736 | +9,167 | +423 |
| Wk-4 | n = 6 | 1,536 | 74,082 | 155,722 | (+14.1%) | (+37.9%) |
| DCV OS | <3 yrs | | | | | |
| Wk-0 | n = 7 | 36,277 | 128,113 | 197,937 | −10,508 | −17,117 |
| Wk-4 | n = 7 | 19,160 | 117,606 | 183,449 | (−8.2%) | (−47.2%) |
| DCV OS | >3 yrs | | | | | |
| Wk-0 | n = 10 | 1,053 | 11,788 | 22,155 | +632 | −254 |
| Wk-4 | n = 10 | 799 | 12,421 | 22,522 | (+5.4%) | (−24.1%) |

Data shown is for all 39 patients. (All) and for two treatment-defined cohorts: TCV = autologous tumor cell vaccine (TCV) (n = 22); DCV = autologous dendritic cell vaccine (DCV) (n = 17). OS = overall survival. Subcohorts were defined by treatment arm and survival greater than (>) or less than (<) three years (3 yrs). Grouped sPD-1 levels were measured at baseline, one week before starting vaccine therapy (wk-0), and four weeks later (wk-4), after the first three vaccine injections.

Baseline sPD-1 Levels were not Prognostic for Survival

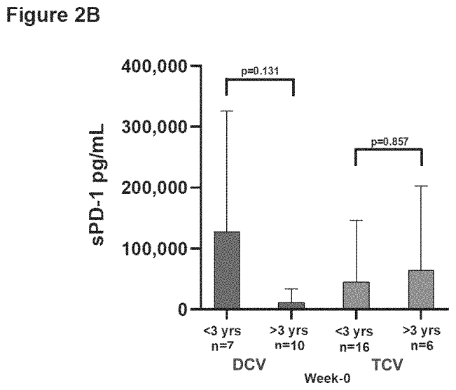

The mean sPD-1 level for three healthy controls was 595 pg/mL, which is in the normal range of 200 to 1200 pg/mL reported by others. For all 39 patients, baseline sPD-1 levels ranged from 0 to 511,063 pg/mL (Tables 1 and 2) with a mean of 54,566 pg/mL and median of 119,845 pg/mL (Table 3). There was no difference in the means of baseline sPD-1 for the 17 DCV-treated compared to the 22 TCV-treated (Table 3, FIG. 2A). There was also no difference in the means of baseline sPD-1 for the 16 patients who survived greater than three years compared to the 23 who survived less than three years (Table 3, FIG. 2A). The most striking variation in Table 3 is the high mean week-0 sPD-1 levels for the seven DCV-treated patients who survived less than three years. This difference is shown graphically in FIG. 2B and contrasted to the low sPD-1 levels recorded at baseline in the 10 DCV-treated patients who survived more than three years, and in TCV-treated patients. However, because of the wide variation in values and the relatively small numbers of patients, this great difference in the mean baseline sPD-1 levels in the 10 DCV-treated patients who survived greater than three years was not significantly different compared to the sPD-1 levels for the 7 DCV-treated patients who survived less than three years (p=0.131) (Table 3, FIG. 2B). Among TCV-treated patients, the median and mean baseline sPD-1 levels were similar for the 16 who survived less than three years and for the six who survived greater than three years (Table 3, FIG. 2B).

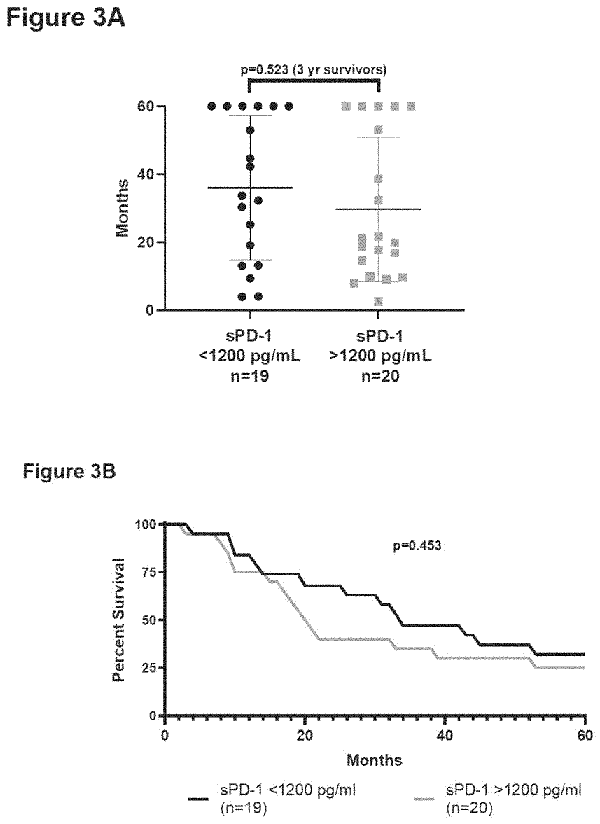

Patients were grouped by sPD-1 levels that were less than or greater than 1,200 pg/mL, which provided the same distribution on either side of the median of 1,509 for all 39 patients (Tables 1, 2, 3). The proportion of patients surviving more than three years was no greater for the 19 patients with baseline sPD-1 less than 1,200 pg/mL compared to the 20 patients with baseline sPD-1 greater than 1,200 pg/mL (9/19 versus 7/20, p=0.523) (FIG. 3A). In addition, overall survival (OS) was no better for 19 patients with baseline sPD-1 less than 1,200 pg/mL compared to 20 patients with sPD-1 greater than 1,200 pg/mL (median OS 33.0 vs 19.9 mos.; 3-yr OS 47% vs 35%, p=0.453) (FIG. 3B). Thus, baseline sPD-1 was not a prognostic marker for survival for this population of melanoma patients.

Changes in sPD-1 Levels from Week-0 to Week-4 were not Predictive of Survival

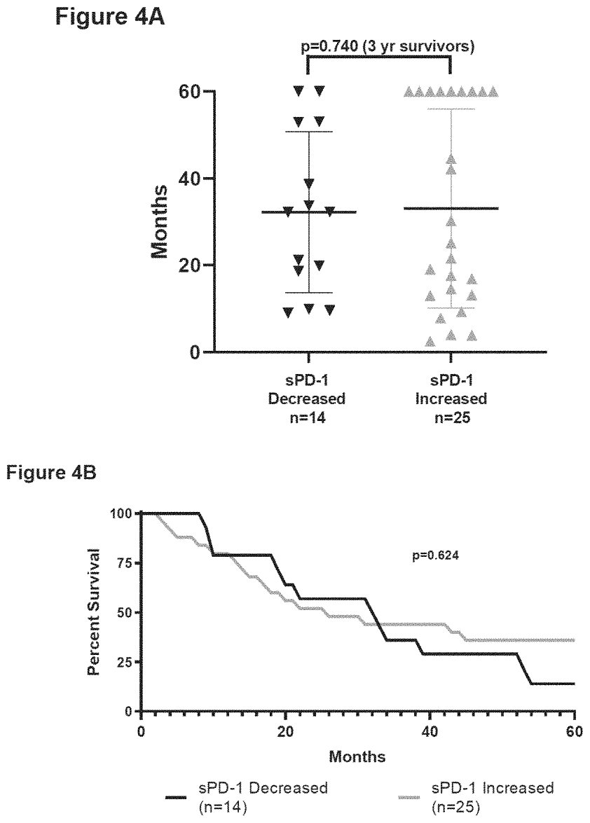

sPD-1 levels decreased in 11 patients and increased in 25 (Tables 1, 2, FIG. 1). Changes in sPD-1 (increased versus decreased) were not predictive of survival as shown by dot plot distribution (FIG. 4A) and survival curves (FIG. 4B). In addition, changes in sPD-1 were not predictive of survival for either treatment arm (Tables 1, 2, FIG. 1A). Changes in sPD-1 levels were also not predictive of survival for the 16 TCV-treated and 7 DCV-treated patients who survived less than three years (Tables 1, 2, FIG. 1B), nor for 6 TCV-treated and the 10 DCV-treated patients who survived more than three years (Tables 1, 2, FIG. 1C). For each of these cohorts, the median and mean levels of sPD-1 were unchanged between week-0 and week-4 (Table 3). Thus, changes in sPD-1 levels were not predictive of survival in either treatment arm or any subset.

Additional Analyses by Treatment Arm Suggested that a Combination of Baseline sPD-1 Levels and Changes in sPD-1 Levels are be Predictive for Survival

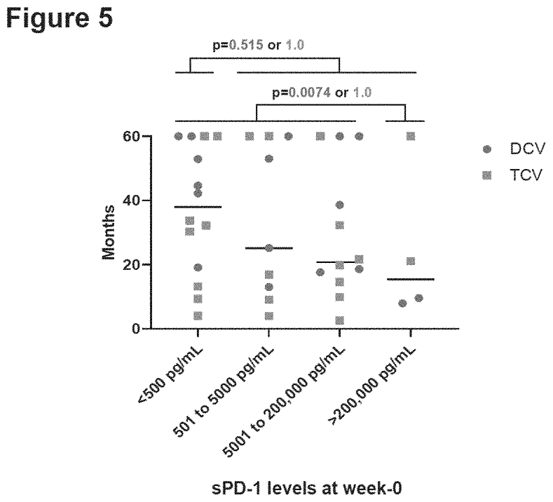

Extremely high baseline sPD-1 levels were associated with poor survival in DCV-treated patients, but not TCV-treated. The association between baseline sPD-1 levels and survival of individual patients for all patients and by treatment are depicted in FIG. 5. There were four patients with baseline sPD-1 levels greater than 200,000 pg/mL (Tables 1, 2, FIG. 5). Two of these patients were DCV-treated; both survived less than 10 months. Two were TCV-treated; one survived 21 months, and the other survived 5 years. The 5-year survivor was a 69-year-old man who entered the study as a recurrent stage 3 patient without measurable disease. He received all eight doses of TCV but progressed with a small bowel metastasis during treatment. This was resected, and during the following year he was treated with ipilimumab followed by resection of another small bowel recurrence. He received no subsequent treatment but remained disease-free the final three years of follow-up.

FIG. 5 shows that all 15 DCV-treated patients with a baseline sPD-1 level of less than 200,000 pg/mL survived more than one year, in contrast to the two DCV-treated patients with a baseline level of greater than 200,000 pg/mL, neither of whom survived a year (15/15 vs 0/2, p=0.0074). Among TCV-treated patients with a baseline sPD-1 less than 200,000 pg/mL, the proportion surviving one year was 14/20 compared to 2/2 for patients with a baseline sPD-1 greater than 200,000 pg/mL (p=1.00) (FIG. 5).

Relatively low levels of sPD-1 were associated with good survival in DCV-treated patients. Among DCV-treated patients, in the cohort with baseline sPD-1 levels less than 500 pg/mL, 5/6 survived more than 3 years compared to 5/11 with baseline sPD-1 levels greater than 500 pg/mL (p=0.304). No such associations were evident for TCV-treated patients. For TCV-treated patients, in the cohort with baseline sPD-1 levels less than 500 pg/mL, 2/8 survived more than 3 years compared to 4/14 with baseline sPD-1 levels greater than 500 pg/mL (p=1.00) (FIG. 5). However, extremely low levels of sPD-1 were associated with good survival in both arms. There were 2/22 patients in the TCV-treated group with baseline sPD-1 levels less than 100 pg/mL; they survived 30 and 60+ months (Table 2). There were 3/17 in the DCV-treated group with such low levels, and their survivals were 42, 45, and 60+ months (Table 1). Thus, the five patients with the lowest baseline sPD-1 levels did relatively well regardless of which vaccine they received. None of these patients ever received anti-PD-1 or anti-PD-L1 therapy.

A decrease in sPD1 levels was associated with longer survival in DCV-treated patients, but not TCV-treated patients. In terms of decreases in sPD-1 levels after three injections, there was no difference in the proportion of DCV-treated patients compared to TCV-treated patients (7/17 vs 7/22, p=0.74) (Tables 1, 2, FIG. 1A). However, among the patients who had a decline in sPD-1 between week-0 and week-4, 5/7 DCV-treated patients survived three years (Table 1), compared to 0/7 TCV-treated patients (p=0.021) (Table 2). This observation combined with the associations of survival with very low and very high baseline sPD-1 levels led to the creation of two cohorts defined by a combination of baseline sPD-1 levels and changes in sPD-1 between week-0 and week-4.

Classification Using a Combination of Baseline sPD-1 and Change in sPD-1 was Predictive of Survival for DCV-Treated Patients

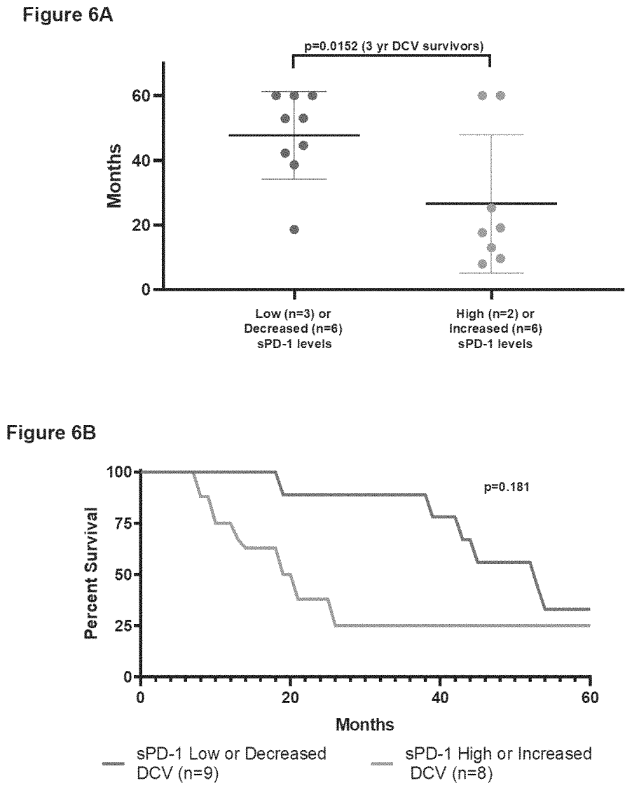

Patients were classified by a combination of baseline sPD-1 levels and change in sPD-1 levels between week-0 and week-4. Specifically, one cohort (sPD-1 low/decreased) was defined by a baseline sPD-1 of less than 100 pg/mL, or less than 200,000 pg/mL but with a decrease in sPD-1 between week-0 and week-4. The second cohort (sPD-1 high/increased) was defined by a baseline sPD-1 of greater than 200,000 pg/mL, or an increase in sPD-1 between week-0 and week-4. FIG. 6 shows the survival of DCV-treated patients individually by dot plot (FIG. 6A) and collectively by survival curves (FIG. 6B), based on these classifications. Those in the sPD-1 low/decreased group had better survival. FIG. 6A shows that 9/17 (52.9%, 95% CI 29.2% to 76.6%) of DCV-treated patients had a baseline sPD-1 level <100 pg/mL, or a week-0 level between 100 pg/mL and 200,000 pg/mL, and a subsequent decrease in sPD-1 levels; their median survival was more than four years with 8/9 surviving beyond three years. In contrast, FIG. 6A also shows that the 8/17 DCV-treated patients in the sPD-1 high/increased group had a median survival of less than two years, and only 2/8 survived beyond three years (p=0.0152). Thus, among the 17 DCV-treated patients, the classification using a combination of baseline sPD-1 and changes in sPD1 correctly classified 8/10 (80%) of 3-year survivors, and 6/7 (86%) of patients who survived less than three years.

The one false positive patient who survived less than 3-years was a 56-year-old female with measurable metastatic disease and elevated lactic dehydrogenase at the time of vaccine treatment. Her sPD-1 was 57,587 pg/mL at baseline and decreased to 19,160 at week-4 (patient #14, Table 1). She received all eight DCV doses, but was found to have progressive disease prior to receiving the last dose. She subsequently was treated with a combination of low-doses of GM-CSF, interleukin-2, and interferon alpha, then a combination of temozolomide, docetaxel, thalidomide, and sorafenib, and finally albumin-bound paclitaxel.

There were two false negative cases, that is, patients for whom the sPD-1 model predicted survival less than three years, but both were still alive five years later. The first was a 46-year-old male who was disease-free after resection of recurrent stage III disease. His sPD-1 was 42,393 pg/mL at baseline and increased to 52,570 pg/mL at week-4 (patient #13, Table 2). Two years after finishing the eight vaccine doses, he had a solitary lung metastasis that was treated with localized intensity modulated high-dose radiation. He remained disease-free thereafter. The other false negative was a 55-year-old male who had experienced local recurrence of ocular melanoma and liver metastases prior to receiving the vaccine, but had no measurable disease at the time of enrollment. His baseline sPD-1 was 7,645 pg/mL and increased to 10,340 pg/mL at week-4 (patient #11, Table 2). He received all eight doses but developed new liver metastases about 10 months after completing the vaccine. He subsequently responded well to high-dose interleukin-2 but progressed, then responded well to anti-PD-1 therapy, but again progressed, and was receiving ipilimumab for progressive disease at the time of 5-year follow-up. He was the only patient among the 39 to have received anti-PD-1 therapy during the course of the trial and follow-up.

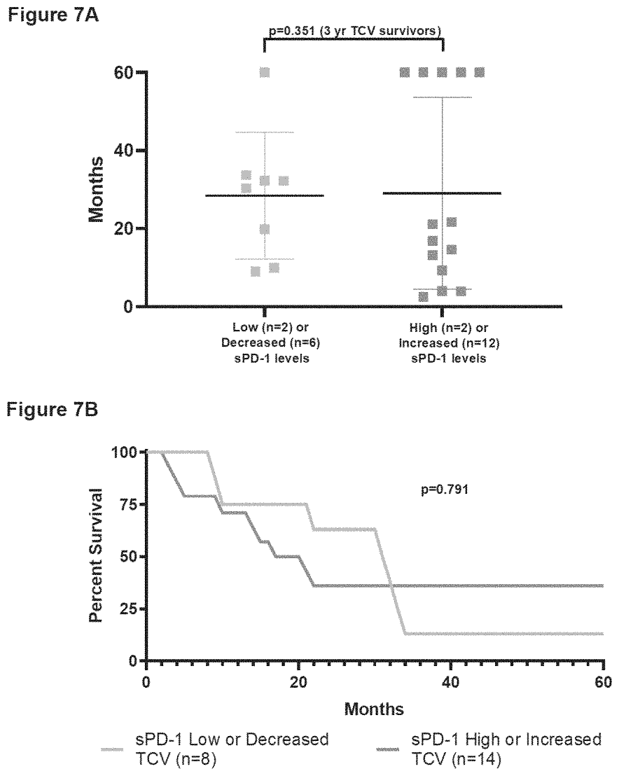

Analysis suggested that TCV-treated patients were more likely to survive three years if their sPD-1 level increased between week-0 and week-4 (5/14) as opposed to decreasing (1/8), but this difference was not significant (p=0.351) (Table 3, FIGS. 1B, 1C). FIG. 7 shows the survival of TCV-treated patients, individually by dot plot (FIG. 7A) and collectively by survival curves (FIG. 7B) based on the same criteria used to classify DCV-treated patients. There were 8/22 TCV-treated patients in the sPD-1 low/decreased cohort. Their probability of surviving more than three years was no different than for those TCV-treated who were classified as sPD-1 high/increased (FIG. 7). In TCV-treated patients, the sPD-1 criteria correctly classified only 1/6 (17%) of 3-year survivors and 10/16 (62%) of patients who survived less than three years.

An interesting aspect of this analysis is the finding that in DCV-treated patients, the “sPD-1 low/decreased” criteria defined by a baseline sPD-1 level less than 100 pg/mL or a baseline level less than 200,000 pg/mL with a subsequent decrease in sPD-1 after three weekly vaccinations, correctly classified 80% of 3-year survivors and 86% of patients who survived less than three years. Similar associations were not seen in TCV-treated patients. In addition to the predictive value of very low or very high baseline sPD-1 levels in DCV-treated patients, in the intermediate range between 100 pg/mL and 200,000 pg/mL, a decrease in sPD-1 levels following vaccination was associated with better survival among DCV-treated patients, but not TCV-treated patients. This is additional evidence that the immunological effects of these two patient-specific vaccines are quite different, and likely explain the differences in survival between the treatment arms.

This data suggests that baseline sPD-1 levels could be useful as a prognostic marker to exclude patients with little chance of benefitting from treatment with DCV, and that combining baseline sPD-1 levels with changes in sPD-1 levels after vaccination could be useful as a predictive marker of survival; and, therefore possibly could be a surrogate marker for survival in trials of metastatic melanoma patients treated with DCV. Having a surrogate marker for survival would be extremely useful for rapidly predicting benefit from DCV therapy in the context of a clinical trial. Excluding patients with very high baseline sPD-1 levels might be useful to select patients that has a better chance of benefitting from such a vaccine.

The findings in this study did not support baseline sPD-1 levels as a prognostic marker for this population of metastatic melanoma patients who enrolled in a clinical trial testing patient-specific autologous cell-based vaccines. For all 39 patients, there was no difference in mean or median levels at baseline or four weeks later after the first three vaccine injections (FIG. 1, Table 3). However, the associations between sPD-1 levels and survival were quite different in DCV-treated patients compared to TCV-treated patients, even though samples were not analyzed for the two TCV-treated patients who had the worst outcome among all patients, nor a DCV-treated patient who was one of the 12 patients in the randomized clinical trial who survived five years. Being classified as sPD-1 low/decreased appeared to have predictive significance for patients treated with DCV, but not TCV. The two DCV-treated patients with the highest levels of sPD-1 at baseline had relatively poor survival.

Patients with no immune response would be expected to have very low levels of sPD-1 and might benefit from an effective vaccine given alone, concurrently with, or preceding treatment with anti-PD-1 or anti-PD-L1. All three DCV-treated patients with a baseline sPD-1 less than 100 pg/mL survived at least 3.5 years, and none received treatment with anti-PD-1 or anti-PD-L1. Patients with a very high sPD-1 level might not benefit from induction of additional immune responses to more antigens, or increased responses to recognized antigens, but might benefit from anti-PD-1 or anti-PD-L1 therapy to unleash the previously suppressed immune responses. Those patients with levels in between the extremes likely would benefit from the combination of a vaccine with immune checkpoint inhibition therapy. There was much better survival in DCV-treated patients, possibly because the ex vivo loading of the cellular antigens onto dendritic cells in DCV provides improved immunogenicity as compared to the TCV.

The data are also relevant because of the potential association between sPD-1 and suppression of immune responses. The levels of sPD-1 and changes in sPD-1 are a reflection of Th1 immune responses and their suppression by the PD-1/PD-L1 axis. A recently induced or augmented immune response may be suppressed by increases in PD-1 expression by T lymphocytes that may result in increases in sPD-1. A decrease in sPD-1 or a lack of increase of sPD-1 following DCV may be indicative of lack of inhibition of a new effective immune response via the PD-1/PD-L1 regulatory axis.

The combination of sPD-1 levels at baseline and change in sPD-1 levels following three weekly vaccines, appeared to predict long-term survival in patients with metastatic melanoma who were treated with autologous DCV.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A method for selecting cancer patients for cancer vaccine therapy comprising, measuring the level of soluble programmed cell death protein-1 (sPD-1) in the blood of the patient, wherein levels of sPD-1 less than about 500 pg/ml are predictive of efficacy of the cancer vaccine therapy.

2. A method for predicting survival of cancer patients after autologous cancer vaccine therapy comprising, measuring a level of sPD-1 in a blood sample from the patient, wherein the measured level of sPD-1 is:
(i) less than about 100 pg/ml before onset of therapy; or
(ii) less than about 200,000 pg/ml before onset of therapy and less after three doses of autologous cancer vaccine therapy than before onset of therapy.

3. A method for treatment of cancer in a patient in need thereof, comprising measuring a level of sPD-1 in a blood sample from the patient; and
administering an autologous cancer vaccine if the level of sPD-1 is less than about 500 pg/ml.

4. A method for treatment of cancer in a patient in need thereof, comprising measuring a level of sPD-1 in a blood sample form the patient before onset of autologous cancer vaccine therapy;
administering one or more doses of autologous cancer vaccine therapy to the patient;
measuring a level of sPD-1 in a blood sample from the patient after the one or more doses of autologous cancer vaccine therapy; and
administering one or more additional doses of cancer vaccine if the level of sPD-1 is less than about 200,000 pg/ml before onset of therapy and the levels of sPD-1 have decreased after one or more doses of autologous cancer patient therapy.

5. A method for treatment of cancer, comprising administering one or more doses of an autologous cancer vaccine therapy to a patient in need thereof wherein the patient has a level of sPD-1 less than about 500 pg/ml.

6. The method of claim 5, further comprising administering an additional dose of the autologous cancer vaccine if the level of sPD-1 in the patient decreased subsequent to the immediately prior administration of the cancer vaccine.

7. A method of treating cancer, comprising administering an autologous cancer vaccine to a patient in need of such therapy, wherein the patient has levels of sPD-1 less than about 500 pg/ml.

8. The method of claim 1, wherein the cancer is melanoma.

9. The method of claim 1, wherein the cancer vaccine is a dendritic cell vaccine.

10. A cancer vaccine for use in a method of treating cancer in a patient who has levels of sPD-1 less than about 500 pg/ml.

11. The method of claim 1 further comprising administering a cancer vaccine therapy if the level of sPD-1 in the patient is less than about 500 pg/ml.

12. The method of claim 1 further comprising administering an immune checkpoint inhibitor therapy if the level of sPD-1 in the patient is not less than about 500 pg/ml.

13. The method of claim 3, further comprising administering an immune checkpoint inhibitor therapy if the cancer vaccine is not administered or an additional dose is not administered.

14. The method of claim 1, wherein the vaccine is autologous.
